(19) United States
(12) Patent Application Publication
(10) Pub. No.: US 2007/0057084 A1
(43) Pub. Date: Mar. 15, 2007

(54) Air-freshener for a vehicle, particularly for a motor vehicle

(76) Inventor: Pedro Vieira, Cascais (PT)

Correspondence Address: MCNAIR LAW FIRM, P.A., P.O. BOX 10827, GREENVILLE, SC, 29603-0827, US

(21) Appl. No.: 11/223,097

(22) Filed: Sep. 9, 2005

(51) Int. Cl. A24F 25/00; A61L 9/04

(52) U.S. Cl. 239/34; 239/47; 239/51.5

(57) ABSTRACT

A vehicle interior air freshener comprises a housing adapted for mounting adjacent to an air outlet through which air flows into a vehicle interior. The housing includes an air admission opening for receiving air from the air outlet into the housing interior; an air outlet opening included in the housing in fluid communication with the air admission opening. A receptacle is carried by the housing containing a scent substance for dispersement into the vehicle interior. A dosing device is carried with the housing for dispensing a predefined scent charge from the receptacle. An intermediate reservoir element is carried between the air admission opening and the air outlet opening so that an air stream colliding with the intermediate reservoir element entrains the scent substance and flows through the air outlet opening. The dosing device being disposed a distance from the reservoir element and manually actuatable to dispense the scent charge onto the reservoir element upon actuation.

### BACKGROUND OF THE INVENTION

The invention is related to an air-freshener for a vehicle, particularly for a motor vehicle.

Air-fresheners are previously known for motor vehicles, for example U.S. 2004/0129742 AI, according to which an air-freshener can be arranged in front of an air outlet nozzle of a ventilating channel system, e.g. an air conditioner, leading into the vehicle interior by means of a holding device. A receptacle containing a liquid scent substance can be screwed into the housing of this air-freshener. The receptacle is equipped with a wick having a lower end extending into the interior of the housing which soaks up the liquid due to its capillary activity. The upper end of the wick protruding from the receptacle is permanently soaked with the scent substance. This air-freshener exhibits a twistable cap-arrangement which contains the wick, and is equipped with a number of air intake and air outlet slits at diametrically opposing sides of the cap. This cap can be twisted in relation to a bearing sleeve, so that, depending on the desire of the operator, the air intake and air outlet slits can be engaged in fluid communication with the interior of the vehicle. A problem occurring very frequently with such wick arrangements is that such air fresheners tend to leak which, in turn, bears the risk of polluting the interior of the vehicle, particularly in the sensitive instrument panel area. A principally similar construction with the same disadvantages is also known from U.S. Pat. No. 4,840,773.

Accordingly, an object of the present invention is to provide an air freshener for a vehicle, particularly for a motor vehicle, by which the leaking of a scent substance into the interior of the vehicle can be avoided in a simple way.

Another object of the invention is to provide an air-freshener for a vehicle, particularly for a motor vehicle, by means of which the intensity of the scent can be adjusted in a simple, as well as functionally safe way.

### SUMMARY OF THE INVENTION

The above objectives are accomplished, according to the present invention, by providing an air-freshener for a vehicle including a housing, preferably installable in front of an air outlet within the vehicle interior. The air outlet delivers a stream of air into the interior of a housing having at least one air outlet opening, in fluid communication with the air admission opening. The air-freshener is equipped with a receptacle for the scent substance to be emitted into the interior of the vehicle. A dosing device conveys and doses a predefined amount of the scent substance from the receptacle onto an intermediate reservoir element as a scent substance charge in batch quantity. This intermediate reservoir element is arranged in such manner in the air stream flowing between the air admission opening and the air outlet opening entrains the scent substance intermediately stored there. The air stream and scent substance exit the interior of the housing via the air outlet opening into the interior of the vehicle as an air/scent substance mixture.

A particular advantage of this air-freshener, according to the invention, is that only predefined quantities of the scent substance are dispensed as a scent substance charge and sprayed into an intermediate reservoir element by means of the dosing device. In a simple way, the intermediate reservoir element, which is preferably made of an absorbent material, is only drenched with an amount of scent substance, to safeguard against leaking of the scent substance from the air-freshener. Spilling of the scent substance inside of the vehicle is most advantageously avoided in a simple way. This permits the operator in a particularly simple way to choose the respective amount of scent substance absorbed by the intermediate reservoir element by individually predefined actuation of the dosing device as is necessary to suit the needs of the operator.

This can, for example, be carried out in a particularly simple and functionally safe way by means of a dosing device that includes a pumping device which dispenses a predefined amount of scent substance fluid per pumping cycle as a scent substance charge sprayed into the intermediate reservoir element. This dosing device is a preferential arrangement for safety reasons, although the dosing device can principally also includes a spraying device. The actuation of the spraying device dispenses very fine droplets of the scent substance, and a predefined amount of the scent substance is expelled from the receptacle per spraying cycle as an aerosol charge. A higher level of safety precautions is, however, required in case of the latter model variant, particularly in connection with the installation of the receptacle, e.g. in order to avoid the unwanted destruction of the receptacle within the interior of the vehicle caused by, for example, too high temperatures. The intermediate reservoir element itself is advantageously produced completely from an absorbent material, e.g. of a cellulose material.

In accordance with another preferential arrangement, the receptacle is held in the housing by means of a releasable holding connection providing for an adjustment space in the housing for the actuation of the dosing device. On the one hand, such a construction ensures that the receptacle can be exchanged and replaced at any time, and on the other hand, that it is safely held in position inside of the housing, when it is mounted. The variable mount insures that a setting range for the pumping movement by the operator, e.g. by exerting manual pressure on the receptacle, is available in a simple manner at any time.

The holding connection can principally be fashioned in a variety of ways, such as a threaded connection. However, a particularly simple and convenient construction of the holding device provides it is formed by a notched connection having a first notched element on the side of the housing and a second notched element attached to the receptacle as a notched counter-element. The notched connection is preferentially constructed as a pressure releasable type notched connection, which permits a particularly simple and fast placement and replacement of the receptacle, e.g., a container for liquids. The notched connection can actually include a ring-shaped projecting part on the housing, which, in locked position, locks in place with a ring-shaped recess on the receptacle, that constitutes a second notched element.

This construction is particularly convenient in such cases, if the ring-shaped projecting part includes a sealing ring, for example, made of an elastomer material. In a locked position with the receptacle the projection part simultaneously seals the housing receptacle area towards the outside, which can provide for additional safety for the prevention of leakage. A form of construction is, however, principally also possible, in which the projecting part is situated in the area of the receptacle, and the recess is positioned on the housing.

For the discharge of batch quantities of the scent substance, the dosing device may be equipped with a spraying valve having a spray nozzle so that a prescribed scent substance charge is dispensed. The scent substance charge is determined by the arrangement and construction of the spraying valve and the intermediate reservoir element wherein the reservoir element is positioned at a predefined distance from the spraying nozzle, and by a tubular shaped dosage tube and spraying nozzle disposed in a nozzle cap in fluid communication with the dosage tube. Depending on the shape of the nozzle, the dispensed liquid scent substance may be atomized, or it may be sprayed onto the intermediate reservoir element as a jet of fluid.

In a mounted position, the receptacle is preferably incorporated in a slide-in placement sleeve of the housing enclosing the receptacle with a positive fit. This ensures correct positioning of the receptacle in a simple and functionally safe way. If such a slide-in placement sleeve is used, then the notched connection described before is located on the inside wall of the slide-in placement sleeve.

The placement sleeve is provided with a slide-in entrance outside of the housing. Within the housing, the sleeve is equipped with a slide-in limiting stop for the receptacle. A passage opening allows the dosage tube at the slide-in limiting stop to protrude through the passage opening in a mounted position. In this arrangement, there is provided a nozzle cap fixed in the passage opening in fluid communication with the dosage tube.

The holding connection of the receptacle in the placement sleeve is designed so that with the receptacle inserted into the placement sleeve, a predefined space is created in the placement sleeve between the limiting stop and the receptacle in its locked position. The spraying valve, upon application of manual pressure applied to the outside end of the receptacle, e.g., a pumping action, is actuated and the scent substance charge is dispensed as a spray dose via the nozzle cap. The pumping action is advantageously performed by relative motion of the receptacle, overcoming the notched connection, in relation to the nozzle cap held in a stationary position. A form of construction would also be possible in which the nozzle cap is moved in relation to the receptacle.

The placement sleeve protrudes into the interior of the housing so that the nozzle of the nozzle cap is proximately aligned with the reservoir element at a predefined distance. This ensures that the reservoir element is wetted completely with the scent substance charge dispensed by spraying the charge in a functionally safe way, without the spillage of any scent substance. In an advantageous embodiment, the reservoir element annularly encloses a section of the placement sleeve protruding into the housing portion containing the nozzle cap. The reservoir element can, for example, also be affixed to the placement sleeve, e.g. slipped onto the placement sleeve with positive fit. The reservoir element can also be, alternatively or additionally, affixed to a retention element of the housing interior positioned at a distance from the placement sleeve and above the nozzle cap. Preferably, the retention element includes a ring-shaped collar support retaining the reservoir element that consists of a strip of flexible material form into a cylinder.

A leak-proof, or almost leak-proof, encapsulation of the nozzle cap within the cylindrical reservoir element is possible with the reservoir element arranged around the nozzle cap. The reservoir element is sealed at its opposing ends in relation to the housing by the collar support on the one hand, and the placement sleeve on the other. This ensures that all spray doses of scent substances emitted by the spraying nozzle are absorbed by the reservoir element, preferably composed of an absorbent material. With this, a particularly high degree of effectiveness for the emission of scent substance.

In accordance with another arrangement of the invention, the nozzle cap is mounted in the housing as a counter-bearing for the actuation of the spraying valve within the collar support. This ensures that the nozzle cap is permanently kept in the mounted position in case of a pumping action of the receptacle, and that it will not come loose by excessive application of pressure.

The air admission opening and the air outlet opening are preferentially arranged on opposing sides of the housing so that the intermediate reservoir element is positioned between the air admission opening and the air outlet opening. This will produce the result that the air admission opening and the air outlet opening are in fluid communication by flow channels on opposite sides of the element. The entering stream of air makes contact with the reservoir element and is divided into first and second air streams for advantageous transmission of the scent substance to the air stream. The streams flow around the intermediate reservoir element, and are reunited in the vicinity of the air outlet opening enriched with scent substance, and dispersed from the air outlet opening into the interior of the vehicle as an air/scent substance mixture.

Adjustment of the intensity of the scent substance emitted into the interior of the vehicle can simply be provided by a closing arrangement whereby a predefined section of the opening is open for transmission, depending on the positioning of the closing arrangement. The closing arrangement preferably consists of a shutter flap(s) hinged to the housing. In principle, the air admission opening can also be closed, alternatively or in addition to the air outlet opening, by such a closing arrangement. This is, for example, suitable in case that no air stream from the air outlet nozzles of the vehicle is to be passed through the air-freshener, but the air outlet nozzles of the vehicle shall furthermore remain active for the fresh air supply into the interior of the vehicle. In such case, the scent substance is only emitted through the air outlet opening due to normal evaporation of the scent substance.

In connection with a closable air outlet opening it is of particular advantage, if this closable air outlet opening is operated in combination with means which prevent the complete closure of the opening, e.g. limiting stops and the like, because this ensures, that unwanted flow conditions will not occur at the air freshener, e.g., an air/scent substance current enriched with scent substance which is directed towards the air outlet nozzle.

The housing can be arranged in front of the air outlet nozzle by means of a holding device arranged in the vicinity of the at least one air admission opening. The holding device is, for example, fashioned in the manner of a clamping fixture affixing the housing to at least one vane of a vane array arranged in the vicinity of the air outlet nozzle. The vanes are preferably arranged so that they are adjustable and closable, thus permitting a further regulation of the intensity of the scent substance-supply into the interior of the vehicle. The adjustment may be combined with one or more shutter flaps at the air-freshener itself, because the intensity of the air current into the air-freshener can be regulated in a simple way by means of such adjustable vanee.

There are principally several options available for the design of the housing, the preferred construction is a housing having of a top shell and a bottom shell connected by means of a notched connection. Such a construction provides for an easy installation and completion of the air-freshener. The housing is preferably produced from a plain and inexpensive plastic material.

### DESCRIPTION OF THE DRAWINGS

The construction designed to carry out the invention will hereinafter be described, together with other features thereof.

The invention will be more readily understood from a reading of the following specification and by reference to the accompanying drawings forming a part thereof, wherein an example of the invention is shown and wherein:

FIG. 1 a schematic side view of an air-freshener according to the invention, which is arranged on a vane of an air outlet nozzle in the interior of a motor vehicle,

FIG. 2 a schematic rendering of the top shell of the air-freshener according to FIG. 1, which is shaped in the form of a helmet, with a strip of cellulose as absorbent intermediate reservoir element, which is shaped to a ring and is ready to be inserted into the top shell,

FIG. 3 a representation corresponding with FIG. 2, with an intermediate reservoir element inserted,

FIG. 4 an interior view of the top shell in accordance with FIG. 3,

FIG. 5 a front view of the top shell in accordance with FIG. 3,

FIG. 6 a rear view of the top shell in accordance with FIG. 3,

FIG. 7 a side view of a bottom shell, which can be connected with the top shell by means of a notched connection, with slide-in placement sleeve for a receptacle,

FIG. 8 an interior view of the bottom shell in accordance with FIG. 7, with the intermediate reservoir element marked in dot-and-dash pattern, and the schematic course of flow through the interior of the housing of an air-freshener.

FIG. 9 a rear view of the bottom shell,

FIG. 10 a schematic crosscut through the bottom shell along the line A-A of FIG. 9, with a nozzle cap prepared for mounting, and a receptacle prepared for introduction,

FIG. 11 a schematic representation according to FIG. 10, with the nozzle cap mounted and the receptacle introduced, and

FIG. 12 a schematic crosscut-clipping through the ready assembled air-freshener in the vicinity of a spraying valve.

### DESCRIPTION OF A PREFERRED EMBODIMENT

Referring now in more detail to the drawings, the invention will now be described in more detail.

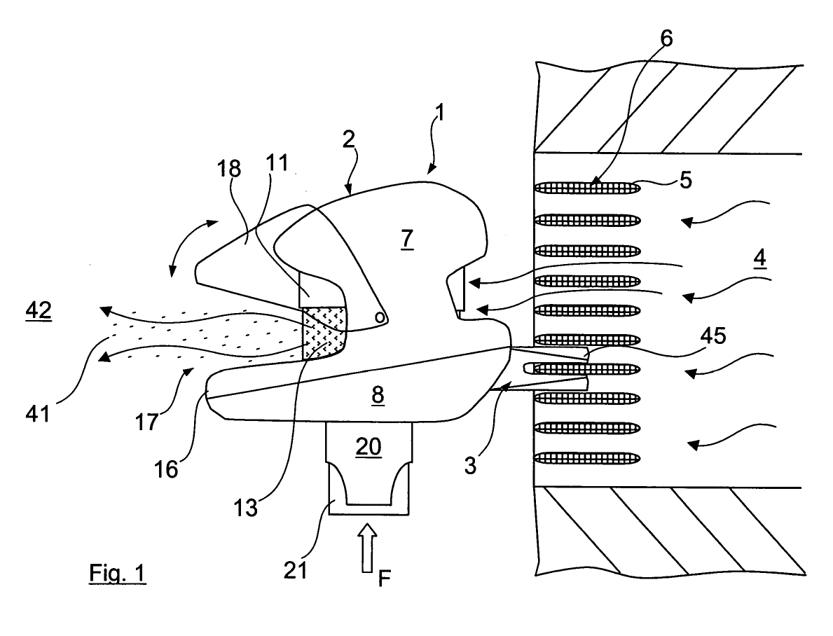

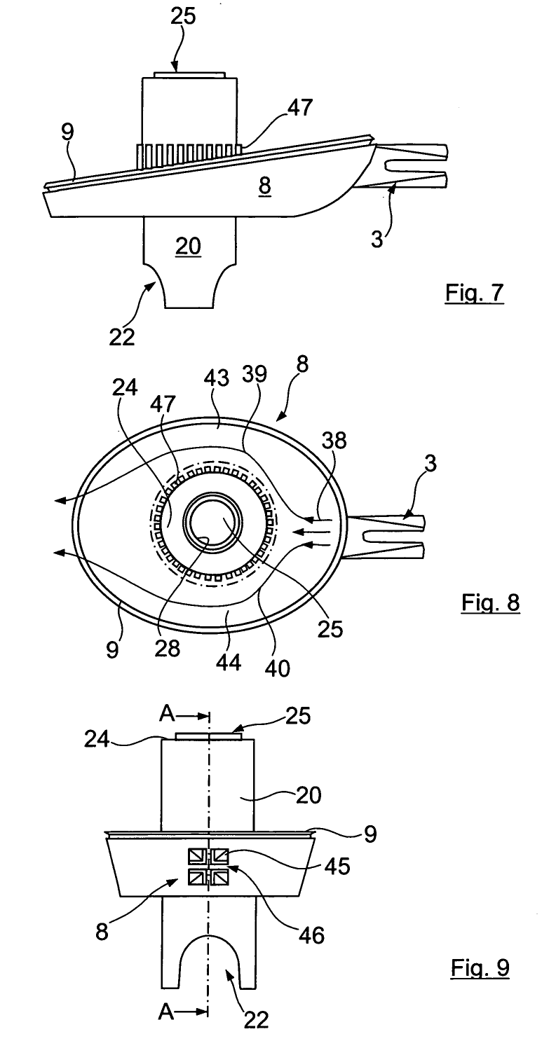

FIG. 1 represents a schematic side view of an air-freshener 1 according to the invention, for a motor vehicle, affixed to a vane 5 of a vane array 6 in the vicinity of an air outlet nozzle 4 by means of a holding device 3 designed as a clamping fixture, and affixed onto a housing 2 of the air-freshener. As may be particularly gathered from FIGS. 1, and 7 to 11, holding device 3 is preferably designed in such manner, so that four elastic clamping fingers 45 form a cross-shaped support 46 for an arrangement on vertical, as well as on horizontal vane of a vane array.

Housing 2 may be made of a plastic material, e.g. polypropylene, and designed in the shape of a motorcycle helmet. In this case, housing 2, may include a top shell 7 and a bottom shell 8 separately connected to each other by means of a fastener or clip connection. As may be seen schematically in FIG. 7, bottom shell 8 is equipped for this purpose with a notched element 9 of the shell running along the rim, which acts in combination with a notched counter-element 10 of shell, represented schematically in FIG. 4.

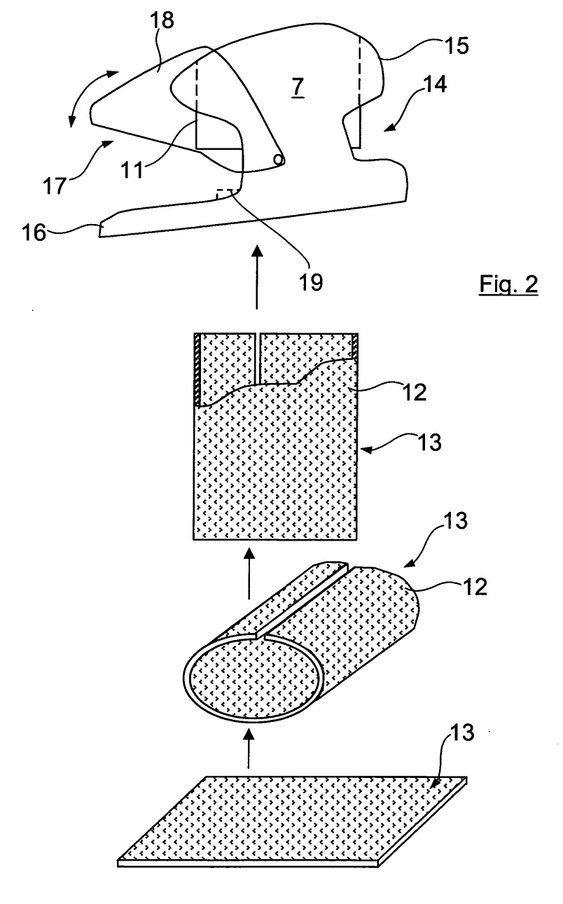

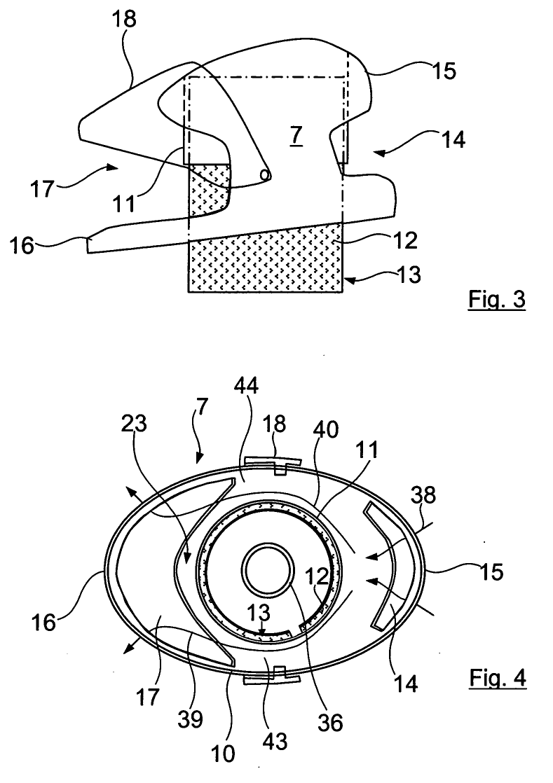

As can best seen in FIGS. 2 to 6, top shell 7 is equipped with a ring shaped collar support 11 for retaining an intermediate reservoir element 12 having the form of a cylinder according to FIG. 3 formed from a rectangular, flexible strip of material. The reservoir element is preferably held in place by frictional engagement, as schematically shown in FIG. 3. Intermediate reservoir element 12 is made from a material 13 displaying high absorptive properties, e.g. a cellulose material.

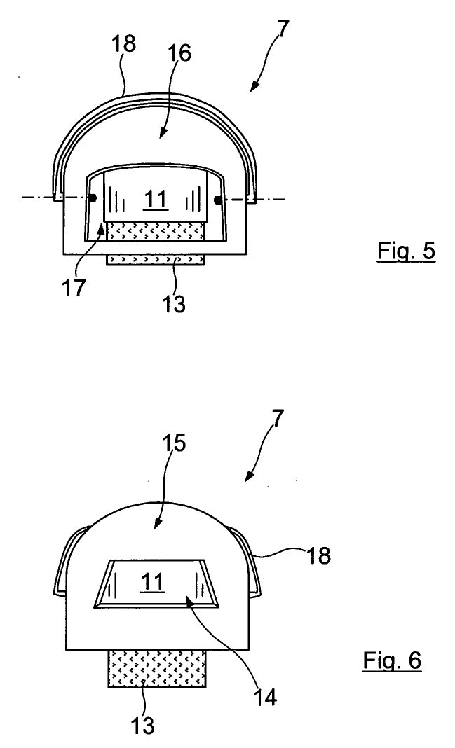

As may be further gathered from the FIGS. 1 to 6, and particularly from the FIGS. 5 and 6, there is an air admission opening 14 at the back side 15 of the top shell 7 aligned and adjacent with air outlet nozzle 4. On a front side 16 of top shell 7, facing air admission opening 14, there is an air outlet opening 17. Absorbent intermediate reservoir element 12 is arranged between air admission opening 14 and air outlet opening 17.

For the purpose of closing air outlet opening 17, top shell 7 has been provided with a shutter flap 18 hinged to opposing lateral areas of the top shell, and designed as a visor. Air outlet opening 17 can control air flow by means of shutter flap 18 to a greater or lesser degree as required by the operator. In this manner, the intensity of scent substance emission can be regulated. The hinged connection of shutter flap 18 is designed so that once adjusted the shutter flap position is maintained. To ensure, that air outlet opening 17 is not closed completely, a small gap in the air outlet opening 17 engaged for ventilation, is provided by a limiting stop 19, preferably disposed on opposite lateral areas of top shell 7, as schematically illustrated in dashed line in FIG. 2.

As can best be seen in FIGS. 7 to 11, bottom shell 8 includes a slide-in placement sleeve 20 for receiving a receptacle 21 containing a liquid scent substance and designed, for example, as a cylindrical container. Slide-in placement sleeve 20 protrudes below the housing to provide a slide-in entrance 22. The sleeve has an upper end extending into the interior of housing 23. At this upper end, slide-in placement sleeve 20 is equipped with a slide-in limiting stop 24. A passage opening 25 is provided in the vicinity of slide-in limiting stop 24 for a spring-loaded dosage tube 26 as a component of a spraying valve 27. Dosage tube 26 protrudes through passage opening 25 and extends into the interior of housing 23 when the receptacle 21 is mounted, as illustrated in FIG. 11.

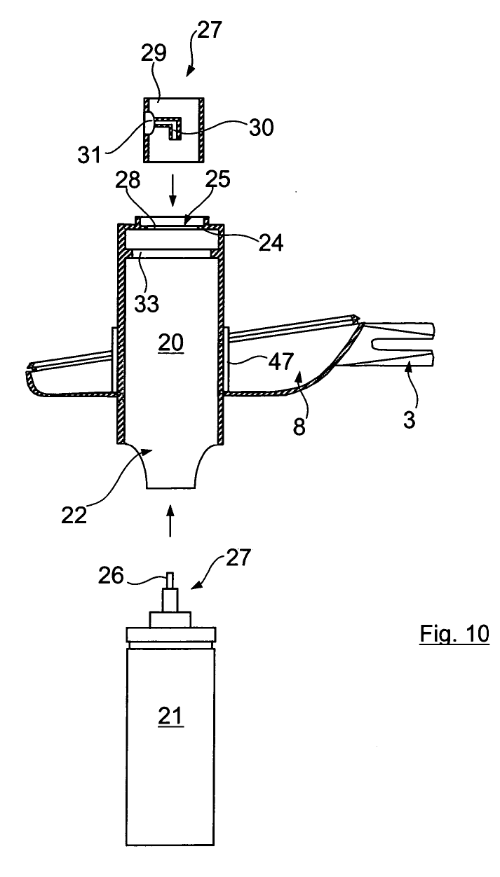

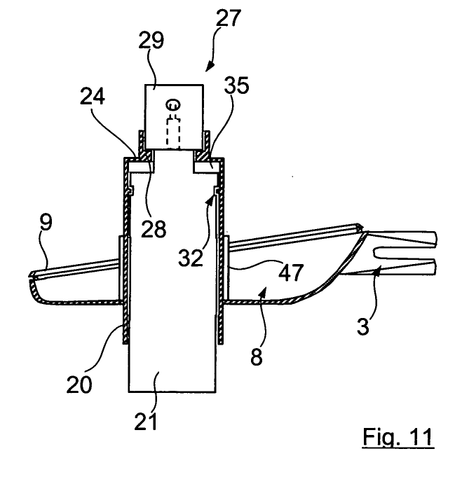

As can best be seen in FIGS. 10 and 11, there is an exemplarily collar mount 28 for affixing a nozzle cap 29 in passage opening 25, forming another component of spraying valve 27. Nozzle cap 29 is preferably provided with a round cross-section profile, and has a cap conduit 30 on the inside, which leads into a nozzle 31 formed in the cap wall. When air-freshener device is mounted, i.e. receptacle 21 is introduced into slide-in placement sleeve 20, and nozzle cap 29 is positioned in passage opening 25, dosage tube 26 is contained in the cap channel 30 with a positive fit and in fluid communication.

A releasable pressure fit is provided by a holding connection in the form of a notched connection 32 to retain receptacle 21 in the slide-in placement sleeve 20. The notched connection includes a ring-shaped projecting part 33 on the sleeve wall. In an interlocked position, as illustrated in FIG. 11, projection part 33 releasably engages a ring-shaped recess 34 on the receptacle providing a counter-notched element. Here projecting part 33 can, for example, be designed as a sealing ring, which engages in the recess 34 with a sealing effect. When inserted in slide-in sleeve 20, receptacle 21 a predefined pumping space 35 is defined between slide-in limiting stop 24 and the receptacle.

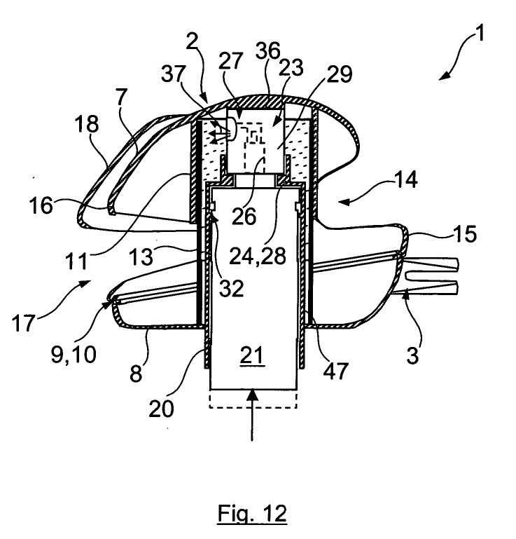

In a ready, assembled condition of air-freshener 1, as best illustrated in FIG. 12, nozzle cap 29 is disposed in top shell 7 in a central area of ring-shaped collar support 11. In this assembled condition nozzle cap 29 protrudes into intermediate reservoir element 12, which annularly encloses nozzle cap 29 and the upper end of slide-in sleeve 20. Thus, nozzle 31 has a predefined spray distance to the inner wall of intermediate reservoir element 12 (FIG. 12). A ribbed structure 47 is furthermore provided here on the outer wall of the slide-in placement sleeve 20, over which the intermediate reservoir element 12 can be slipped for retention.

Spraying valve 27 of the pumping device provides a dosing device by which a scent substance charge amount may be dispensed. The charge amount, predefined by the construction and lay-out of the spraying valve and the receptacle, as well as of the spray distance, is pumped from receptacle 21 per pumping cycle as a scent substance charge, and sprayed onto intermediate reservoir element 12 as a spray dose 37, which absorbs the scent substance charge. To actuate the pump and spray, receptacle 21 is pressed upwards into stationary fixed housing 2 by the operator according to is indicator arrow F. As a result, receptacle 21 overcomes and is released from notched connection 32 and is displaced along the pumping space 35, as the maximum displacement path. Spring-loaded dosing tube 36, fixed in cap conduit 30 of the nozzle cap, is pushed downwardly into the receptacle 21 against an elastic force of a spring imposed upon it, releasing spray dose 37 in a manner as is in principle commonly known. The receptacle is then pulled downwardly until it once again is held by notched connection 32. This pumping cycle can be repeated by the operator as often as needed until a sufficient scent substance amount has been absorbed by the intermediate reservoir element 12. The desired scent substance amount can therefore be dosed exactly by this charge-like pumping action.

As is best seen from FIGS. 1, 4 and 8, an air stream 38 flowing through the vane array 6 will arrive at air admission opening 14 in the vicinity of the housing 2, and through there, it will access the interior of the housing 23. After entering the housing 2, air stream 38 makes contact with the intermediate reservoir element 12, and is divided into a first partial air stream 39 and a second partial air stream 40. The air streams flow circumferentially around the intermediate reservoir element, and are reunited in the vicinity of air outlet opening 17. The air streams, enriched with scent substance 41, are delivered through air outlet opening 17 into the interior of vehicle 42, as illustrated schematically in FIG. 1. The scent substance 41 is, for example, distributed as an aerosol in the air flowing into the interior of the vehicle 42. Partial air streams 39 and 40 thereby flow in flow channels 43, 44, which link the air admission opening and the air outlet opening on opposite sides of the intermediate reservoir element 12 in fluid communication.

While a preferred embodiment of the invention has been described using specific terms, such description is for illustrative purposes only, and it is to be understood that changes and variations may be made without departing from the spirit or scope of the following claims.

1. A vehicle interior air freshener comprising:
a housing adapted for mounting adjacent an interior air outlet of the vehicle through which air flows into an interior of the vehicle;
said housing including an air admission opening for receiving air from said interior air outlet directed into the interior of said housing;
an air outlet opening included in said housing in fluid communication with said air admission opening;
a receptacle containing a scent substance for dispersement into the vehicle interior;
a dosing device for dispensing a predefined scent charge of the scent substance from the receptacle;
an intermediate reservoir element carried between said air admission opening and said air outlet opening for receiving a dose of said scent charge from the dosing device so that a stream of air colliding with the intermediate reservoir element entrains the scent substance and escapes from the housing through said air outlet opening into the vehicle interior for freshening the air of the vehicle interior.

2. The device of claim 1 wherein said dosing device includes a pumping device for dispensing said predefined scent charge during a fluid pumping cycle from said receptacle onto the intermediate reservoir element.

3. The device of claim 1 wherein said dosing device includes a spraying device for propelling said scent charge as very fine droplets expelled from the receptacle per spraying cycle as an aerosol charge.

4. The device of claim 1 wherein said intermediate reservoir element is formed at least in part by an absorbent material.

5. The device of claim 4 wherein said intermediate reservoir element includes a cellulose material.

6. The device of claim 1 including a holding connection for removably mounting said receptacle in said housing, said holding connection having a motion range providing relative movement between said receptacle and housing for the actuation of said dosing device.

7. The device of claim 6 wherein said holding connection includes a notched connection having a first notched element carried by the housing and a second notched element carried by the receptacle.

8. Air-freshener of claim 7 wherein said notched connection is constructed and arranged as a releasable notched connection overcomeable by force on the receptacle; and
said first notched element is formed by a radial projection found on said housing, said second notched element including a recess formed on said receptacle, and said notched connection having a locked position in which said projection is received within said recess.

9. The device of claim 1 said receptacle includes a dispensing element forming part of said dosing device.

10. The device of claim 9 wherein said dosing device includes a spraying valve having a spraying nozzle for dispensing said predefined scent charge.

11. The device of claim 10 wherein said scent charge amount is predefined by a distance from the spraying nozzle to said intermediate reservoir element, and by said dispensing element provided in the form of a tubular shaped dosage tube in fluid communication with a nozzle cap of said spraying nozzle.

12. The device of claim 1 including a slide-in placement sleeve carried by the housing for receiving said receptacle in a mounted position.

13. The device of claim 12 wherein said placement sleeve includes a slide-in entrance on a lower end of said sleeve for receiving said receptacle, and said sleeve includes a slide-in limit on an opposing end of said sleeve for limiting upward movement of said receptacle.

14. The device of claim 13 including a passage opening for the dosage tube adjacent said slide-in limiting stop so that said dosage tube protrudes through said passage opening in the mounted position.

15. The device of claim 14 including a nozzle cap disposed above said passage opening;
said nozzle cap being in fluid communication with the dosing tube so that when said receptacle is in said mounted position, a predefined space between said receptacle and nozzle cap so that the scent charge is dispensed as a spray dose upon actuation of said spray nozzle.

16. The device of claim 15 wherein said placement sleeve protrudes into the interior of the housing so that a nozzle of said nozzle cap is proximately aligned at a predefined distance from said reservoir element.

17. The device of claim 13 wherein said intermediate reservoir element encloses at least part of said placement sleeve.

18. The device of claim 17 wherein said placement sleeve is carried within said reservoir element.

19. The device of claim 18 including a retention element carried by said housing above said nozzle cap for retaining said reservoir element.

20. The device of claim 19 wherein said retention element includes a collar support and said reservoir element includes a tubular element comprised of flexible material.

21. The device of claim 20 wherein said nozzle cap is carried by said housing and provides a bearing surface against which said receptacle may be urged.

22. The device of claim 21 wherein said nozzle is disposed within said collar support and surrounded by said reservoir element.

23. The device of claim 22 wherein said air admission opening and air outlet opening are arranged generally on opposing sides of said housing, and said intermediate reservoir element is disposed between said air admission opening said air outlet opening so that an entering air stream of air contacts said intermediate reservoir element and is divided into a first partial air stream and a second partial air stream which flow around said reservoir element and are reunited in the vicinity of said air outlet opening enriched with the scent substance before flowing from the air outlet opening into the vehicle interior.

24. The device of claim 1 wherein one of said outlet openings and said air admission opening can be closed by closing arrangements so that only a predetermined portion of said opening transmits an air stream.

25. The device of claim 24 wherein said closing arrangement includes shutter flaps hinged to said housing.

26. The device of claim 25 including limit means which prevent the complete closure of said opening.

27. The device of claim 1 including holding device for mounting said housing adjacent said air admission opening.

28. The device of claim 26 wherein said holding device includes a clamping fixture for attaching said housing to at least one vane of an air of an air directing vane array of an air outlet nozzle.

29. The device of claim 28 wherein said vane is adjustable and closable.

30. The device of claim 1 wherein said housing includes a top shell and a bottom shell, and connector means for connecting said top and bottom shell together.

31. A vehicle interior air freshener comprising:
a housing adapted for mounting adjacent to a vehicle air outlet of the vehicle through which air flows into an interior of the vehicle;
said housing including an air admission opening for receiving air from said vehicle air outlet into the interior of said housing;
an air outlet opening included in said housing in fluid communication with said air admission opening;
a receptacle carried at least partially in said housing containing a scent substance for dispersement into the vehicle interior;
a dosing device carried with said housing for dispensing a predefined scent substance from the receptacle as a scent charge;
an intermediate reservoir element carried between said air admission opening and said air outlet opening and
said dosing device being disposed a distance from said reservoir element and being manually actuatable to dispense said scent charge onto said reservoir element upon actuation so that an air stream colliding with the intermediate reservoir element entrains the scent substance of said housing and flows through said air outlet opening into the vehicle interior for freshening the air of the vehicle interior.

32. The device of claim 31 wherein said dosing device includes one of a pumping device and spraying device for dispensing said predefined scent charge during a charge cycle from said receptacle onto the intermediate reservoir element.

33. The device of claim 31 wherein said intermediate reservoir element is formed at least in part by an absorbent material.

34. The device of claim 31 including a releasable holding connection movably mounting said receptacle in said housing having a motion range providing relative movement between said receptacle and housing for the manual actuation of said dosing device.

35. The device of claim 31 including a slide-in placement sleeve carried by the housing for receiving said receptacle in a mounted position, said placement sleeve including a slide-in entrance on a lower end of said sleeve for receiving said receptacle, and said sleeve including a slide-in limit on an opposing end of said sleeve for limiting upward movement of said receptacle relative to said sleeve.

36. The device of claim 35 including a dosing tube carried by said receptacle, a nozzle cap disposed above said dosing tube, and said nozzle cap being in fluid communication with said dosing tube so that said scent charge is dispensed through said dosing tube and said nozzle cap upon actuation of the dosing device.

37. The device of claim 36 wherein said intermediate reservoir element encloses at least part of said placement sleeve and nozzle cap.

38. The device of claim 37 wherein said nozzle cap is carried by said housing in a fixed position, said receptacle is held by said releasable connection to define a space between said nozzle cap and said receptacle in a locked position, and said nozzle cap provides a bearing surface against which said receptacle is urged upon manual actuation upwards to generally close said space whereby said dosing device is actuated.
